(19) United States
(12) Patent Application Publication
(10) Pub. No.: US 2005/0249665 A1
(43) Pub. Date: Nov. 10, 2005

(54) Urea-, glycerate- and, hydroxyamide-headed hydrocarbon chain lyotropic phases forming surfactants

(75) Inventors: Benjamin James Boyd, Warrandyte (AU); Gregory Andrew Davey, Burwood (AU); Calum John Drummond, Rozelle (AU); Celesta Fong, Highett (AU); Patrick Gordon Hartley, Malvern (AU); Irena Krodkiewska, Cheltenham (AU); Annette Joan Murphy, Glen Iris (AU); Russell John Tait, Deepdene (AU); Gregory Goodman Warr, Earlwood (AU); Darrell Wells, Rowville (AU); Darryl Vanstone Whittaker, Vermont (AU); Yuerong Rose Ye, Osmond (AU)

Correspondence Address: BIRCH STEWART KOLASCH & BIRCH, PO BOX 747, FALLS CHURCH, VA, 22040-0747, US

(73) Assignee: DBL Australia Pty Ltd., Melbourne (AU)

(21) Appl. No.: 11/071,113

(22) Filed: Mar. 4, 2005

Related U.S. Application Data
(63) Continuation of application No. PCT/AU03/01139, filed on Sep. 4, 2003.

(30) Foreign Application Priority Data
Sep. 5, 2002 (AU) 2002951216

(51) Int. Cl. A61K 49/00; A61K 7/42; A61K 48/00; A61K 9/127; C07C 235/04

(52) U.S. Cl. 424/9.1; 424/59; 554/61; 564/32; 424/450

(57) ABSTRACT

The invention provides a compound containing a head group based on urea, glycerol or glycerate and a tail selected from the group consisting of a branched alkyl chain, a branched alkyloxy chain or an alkenyl chain. The compounds may be used as surfactants to form a lyotropic phase that is stable in excess polar solution.

### FIELD OF THE INVENTION

The present invention relates to novel surfactants, and also to novel surfactants that are able to form reverse lyotropic phases in aqueous solution.

### BACKGROUND OF THE INVENTION

Surfactants are amphiphilic compounds that contain a charged or uncharged polar region and a hydrocarbon or fluorocarbon non-polar region. The hydrophilic polar and hydrophobic non-polar regions are often termed the head group and tail respectively in linear shaped surfactants.

Due to the amphiphilic character of these materials, the head group tends to associate with polar solvents such as water, and the tails tend to associate with hydrophobic materials, such as oils, or the hydrocarbon tails of other surfactant molecules. Thus, the surfactants tend to reside at the interface between hydrophilic and hydrophobic domains in a mixture of the surfactant with water and other components, as this is the most energetically favourable environment. This surface activity has led to such amphiphilic compounds being known in the art as surfactants, a contraction of surface active agents.

Addition of water to surfactant materials results in water being incorporated into the structure, with the water being associated with the head groups. Incorporation of water into a neat surfactant leads to fluidity in the hydrophilic domains of the mixture, allowing the native geometry of the surfactant molecule to determine the orientation, and spatial aspects of arrangement of molecules at the interface. This arrangement is often called the ‘curvature’, because depending on the relative volumes of the headgroup and tail sections of the molecule, and the relative volumes of water and surfactant, the interface will be curved towards the water or oil sections. The addition of greater amounts of water to the surfactant will alter the average curvature in the system, resulting in a variety of particular geometries that can be adopted in the system at equilibrium. At equilibrium, these particular geometries are often termed ‘mesophases’, ‘lyotropic phases’ or just ‘phases’.

The combination of partial order and partial freedom of the surfactants in ordered phases is reminiscent of classical liquid crystals, and hence these phases are often referred to also as liquid crystalline phases. In these phases most of the order of a crystalline solid is lost and the surfactant molecules are also able to move, unlike molecules in a solid crystal. Hence these types of systems are often referred to as a liquid crystal. Liquid crystalline phases that form in mixtures of amphiphile and solvent (usually water) may also be known as ‘lyotropic liquid crystalline phases’.

Additionally, if the average curvature of a surfactant-solvent system is towards oil, then the mesophases are usually identified as being ‘water-continuous’ and of the ‘normal’ type. If the curvature is towards water, they are termed ‘oil-continuous’ and are said to be of the ‘reverse’ or ‘inverse’ type. If the average curvature is balanced between the two, the system has an average curvature close to zero, and the resulting phases may be of a stacked lamellar-type structure, or a structure often termed ‘bicontinuous’, consisting of two intertwined, continuous, hydrophilic and hydrophobic domains.

Examples of the particular geometries that can be formed in surfactant-solvent systems include reverse micellar, reverse hexagonal, lamellar, reverse cubic, bicontinuous cubic, normal cubic, normal hexagonal and micellar, among others. Micelles occur when surfactant molecules self-assemble to form aggregates due to the headgroups associating with water, and the tails associating with other tails to form a hydrophobic environment. Normal micelles consist of a core of hydrophobic tails surrounded by a shell of headgroups extending out into water.

Addition of further water to this system dilutes the micelles, and depending on the solubility of the surfactant molecules in water, a greater or lesser dilution will result in breakdown of the aggregate to form a solution of monomeric surfactant in water. Addition of a non-water soluble oil will result in some oil being incorporated (or solubilized) into the hydrophobic interior core of the micelles, until a limit in the capacity is reached. Addition of further oil leads to the formation of a separate oil phase excluded from the micellar solution, and the system is said to be phase separated. Reverse micelles are directly analogous to the normal micelles except that the core of micelle contains water in association with the headgroups and the tails extend into a hydrocarbon-continuous domain. Addition of oil dilutes the micelles as discrete entities, and addition of water ‘swells’ the micelles until the capacity of the core to solubilize water is exceeded, resulting in phase separation. The micelles themselves may be spherical, rod-like or disk shaped, depending on the molecular geometry of the surfactant, but are at low enough concentration that the system is essentially isotropic.

Normal hexagonal phase occurs when the system consists of long, rod-like micelles at very high concentration in water, packed into a hexagonal array. As such the system possesses structure in two dimensions. This imparts an increased viscosity on the system, and the anisotropy allows visualisation of the birefringent texture when viewed on a microscope through crossed polarising filters. Again, reverse hexagonal phase is the oil continuous version of the normal hexagonal phase, with water-core micelles in a close packed hexagonal array.

Lamellar phase consists of a stacked bilayer arrangement, where opposing monolayers of headgroups are separated by the water domain to form the hydrophilic layer, while the tails of the back to back layers are in intimate contact to form the hydrophobic layer. This phase is favoured when the surfactant geometry is such that the relative volumes of hydrophobic and hydrophilic regions of the molecule are close to equivalent.

Cubic phase consists of two main types, bicontinuous and micellar. Normal and reverse cubic phases are of the micellar type, and are analogous to the hexagonal phases, in that they consist of close packed spherical micelles in a cubic array, where either the water and headgroups, or the tails form the interior of the micelles. They are generally of high viscosity, but because they consist of spherical micelles these systems are isotropic, so no birefringent texture is observed. Bicontinuous phases form when the molecular geometry of a surfactant molecule is well balanced, such that the curvature is zero. This results in a so-called ‘infinite periodic lattice structure’, in which the hydrophobic and hydrophilic domains are intertwined but do not intersect. For the purposes of this invention bicontinuous phases may be included under the terminology ‘reverse lyotropic phase’, ‘reverse lyotropic phases’, or ‘reverse liquid crystalline phases’.

The order in which these lyotropic phases occur with increasing water to surfactant ratio is definite. As eluded to above, a typical progression of mesophases encountered for a surfactant with increasing amounts of water added could be reverse micellar, reverse hexagonal, lamellar, reverse cubic, bicontinuous cubic, normal cubic, normal hexagonal and micellar. It is important to realise that not all phases may be observed upon dilution for a particular surfactant, but the order of the phases is retained.

For some surfactants, the geometrical constraints may be such that no normal type phases are formed at all. In this case a reverse lyotropic phase, or a lamellar phase may only swell with water up to a certain point, beyond which no more water is incorporated, and a phase separation occurs. In these cases the phase is said to be in equilibrium with excess water and importantly is said to be ‘stable to dilution’. In theory, it is possible with these systems to fragment the water-saturated lyotropic phase to form a particulate dispersion of the material down to the colloidal size range.

In the case of lamellar phase in excess water, imparting energy into the system allows fragmentation of the bilayer structure, upon which the ‘ends’ of the fragments may join together to form a spherical bilayer particle, entrapping a pocket of water inside the bilayer sphere. These types of particles are often termed a vesicle. If the bilayer forming material is a lipid such as di-acyl phosphatidyl choline, the term ‘liposome’ is often used. Depending on the energy imparted on the system, and the method of manufacture, multilamellar vesicles and/or unilamellar vesicles may exist in solution. These types of systems are reasonably common, and due to their membrane-like structure, form the basis of many intracellular processes. However the formation of these structures is not exclusively exhibited by endogenous materials, and many synthetic surfactants with appropriate molecular structure can also form a lamellar phase that is stable to dilution.

Less common are surfactants that form true reverse phases, such as reverse hexagonal phase, or cubic phases, that are also stable to dilution. Analogous to the di-acyl phosphatidyl choline system, di-acyl phosphatidyl ethanolamine with certain acyl chain lengths is known to form reverse hexagonal phase that is stable to dilution. Glycolipids with two phytanyl chains have also been reported to form reverse hexagonal phase in excess water. In these cases, the reverse phase saturated with water can also be fragmented to form particles of hexagonal phase stable in excess water, which have been termed hexosomes.

Even less common is the occurrence of surfactants that form bicontinuous cubic phases that are stable in excess water. Glycerol monooleate is one such surfactant, as is phytantriol. Again a dispersion of the water-saturated bulk phase can be dispersed with the input of energy to form a particulate dispersion that is stable in excess water. The particles in this case have been termed cubosomes.

It should be noted that dispersed particles such as liposomes, cubosomes and hexosomes are not thermodynamically stable and will flocculate over time back to the original bulk phase separated reverse phase and excess water. This can be prevented in some instances by addition of surface stabilisers, which provide a barrier to prevent flocculation.

The potential use of surfactants which form normal phases are well described, and include detergency either by solubilization of oily soils or by substrate surface modification, lubrication, production and stabilisation of foams, stabilisation of emulsions, the wetting of powders for ease of production and enhanced dissolution rates, among many others.

Reverse lyotropic phases are often highly viscous, a property that makes these materials particularly useful in applications where the immobilisation of a particular agent is of importance. The ability to manipulate the phase behaviour to produce low viscosity phases where required, through subtle changes to the composition of the system, or to other variables, such as temperature, exemplifies the usefulness of compositions prepared from these type of surfactants. The potential uses of surfactants that form reverse lyotropic phases that are stable in excess water would be of particular relevance to processes where dilutability is a critical aspect. Also, the use of reverse lyotropic phases in the biomedical field for the immobilisation of membrane proteins has already been described using a glycerol monoolein cubic phase. However, there is a need for systems that enable the study of membrane proteins that not suited to the dimensional aspects of the cubic phase formed by glycerol monoolein. In addition, the working temperature range of the glycerol monoolein system is restricted and this limits the range of applications in which the system can be used.

### SUMMARY OF THE INVENTION

The present invention arises out of the discovery of new classes of surfactants that form reverse lyotropic phases in aqueous solution. The reverse lyotropic phases may be of the micellar type, or of the various liquid crystalline types, such as reverse hexagonal, or bicontinuous cubic phases. The formation of reverse lyotropic phases is principally a function of the structure of the amphiphile. In particular, amphiphiles having a combination of a relatively small polar head group and a tail that occupies a wedge or conical shaped space in solution tend to form reverse lyotropic phases in excess aqueous solution.

Accordingly, the present invention provides a compound containing a head group selected from the group consisting of any one of structures (I) to (V): 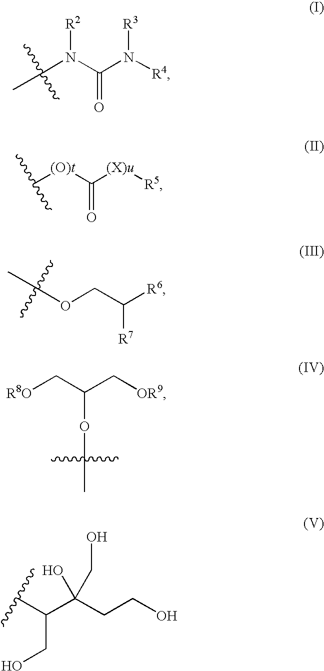
and a tail selected from the group consisting of a branched alkyl chain, a branched alkyloxy chain or an alkenyl chain, and wherein
in structure (I) R² is —H, —CH₂CH₂OH or another tail group,
   R³ and R⁴ are independently selected from one or more of —H, —C(O)NH₂, —CH₂CH₂OH, —CH₂CH(OH)CH₂OH,
in structure (II) X is O, S or N,
   t and u are independently 0 or 1,
   R⁵ is —C(CH₂OH)₂alkyl, —CH(OH)CH₂OH (provided the tail group is not oleyl), —CH₂COOH,
   —C(OH)₂CH₂OH, —CH(CH₂OH)₂, —CH₂(CHOH)₂CH₂OH,
   —CH₂C(O)NHC(O)NH₂,
in structure (III) R⁶ is —H or —OH,
   R⁷ is —CH₂OH or —CH₂NHC(O)NH₂,
in structure (IV) R⁸ is —H or -alkyl,
   R⁹ is —H or -alkyl.

Preferably, the tail is selected from: 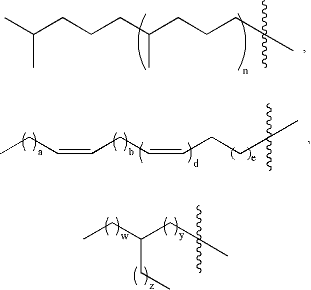
wherein n is an integer from 2 to 6, a is an integer from 1 to 12, b is an integer from 0 to 10, d is an integer from 0 to 3, e is an integer from 1 to 12, w is an integer from 2 to 10, y is an integer from 1 to 10 and z is an integer from 2 to 10.

The present invention also provides a surfactant which is capable of forming a reverse lyotropic phase in excess aqueous solution, the surfactant containing a head group selected from the group consisting of any one of structures (I) to (V): 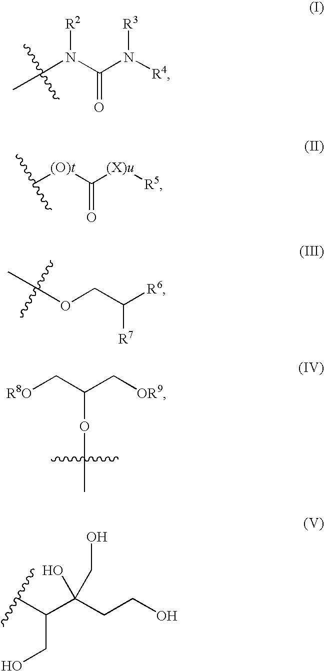
and a tail selected from the group consisting of a branched alkyl chain, a branched alkyloxy chain or an alkenyl chain, and wherein
in structure (I) R² is —H, —CH₂CH₂OH or another tail group,
   R³ and R⁴ are independently selected from one or more of —H, —C(O)NH₂, —CH₂CH₂OH, —CH₂CH(OH)CH₂OH,
in structure (II) X is O, S or N,
   t and u are independently 0 or 1,
   R⁵ is —C(CH₂OH)₂alkyl, —CH(OH)CH₂OH (provided the tail group is not oleyl), —CH₂COOH,
   —C(OH)₂CH₂OH, —CH(CH₂OH)₂, —CH₂(CHOH)₂CH₂OH,
   —CH₂C(O)NHC(O)NH₂,
in structure (III) R⁶ is —H or —OH,
   R⁷ is —CH₂OH or —CH₂NHC(O)NH₂,
in structure (IV) R⁸ is —H or -alkyl,
   R⁹ is —H or -alkyl.

Preferably, the tail is selected from: 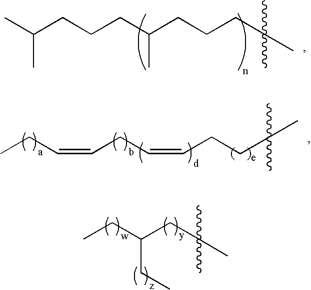
wherein n is an integer from 2 to 6, a is an integer from 1 to 12, b is an integer from 0 to 10, d is an integer from 0 to 3, e is an integer from 1 to 12, w is an integer from 2 to 10, y is an integer from 1 to 10 and z is an integer from 2 to 10.

Under suitable conditions, the surfactants of the present invention form thermodynamically stable reverse lyotropic phases in excess water. Preferably, the lyotropic phase that is formed is selected from the group consisting of a reversed micellar phase, a bicontinuous cubic phase, a reversed intermediate liquid crystalline phase and a reversed hexagonal liquid crystalline phase. Most preferably the reverse lyotropic phase that is formed is a bicontinuous cubic liquid crystalline phase or a reversed hexagonal liquid crystalline phase. These phases are all well characterised and well established in the field of mesomorphism of surfactants.

The present invention also provides a composition containing a reverse lyotropic phase formed from a surfactant of the present invention. The reverse lyotropic phases may be in the form of a colloidal dispersion and accordingly the present invention also provides a colloidal particle consisting of a reverse lyotropic phase of the micellar or liquid crystalline type, formed from a surfactant of the present invention.

### DETAILED DESCRIPTION OF THE INVENTION

The present invention results from the discovery of a novel class of urea-based compounds that were shown to form reverse lyotropic hexagonal phases in excess water at elevated temperatures. The present invention arises out of that discovery and also further work to create surfactants that would form these phases at lower temperatures. The creation of reverse micellar, reverse hexagonal or cubic phases at lower temperatures allowed the formation of preparations containing such reverse phases that were stable at ambient temperature and therefore were commercially useful.

Based on urea, glycerol or glycerate headed surfactants, a number of compounds were synthesised and their behaviour in aqueous solutions was studied. In screening new compounds for phase behaviour, it was found that there was a crude correlation between the melting point of the neat compound, and the temperature range at which a reverse lyotropic phase formed in water. Notably, the lower the melting point of the pure compound, the lower the temperature at which a reverse lyotropic phase was formed. As discussed, commercially those surfactants that form a reverse lyotropic phase in water at temperatures less than about 150° C. were deemed to be most suitable, although it will be appreciated that the invention is not limited to surfactants and reverse lyotropic phases that form only within this preferred temperature range.

Surfactants of the present invention having any one of the head groups shown in Table 1 have either been synthesised and demonstrated to specifically form or are expected to form reverse lyotropic phases in excess water based on data obtained from the surfactants that have been synthesised to date.

TABLE 1

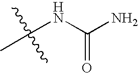

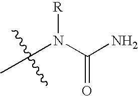

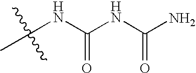

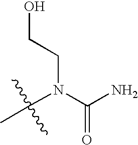

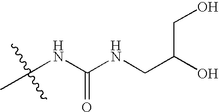

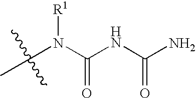

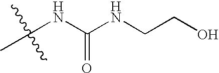

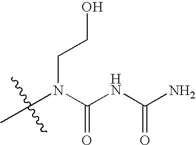

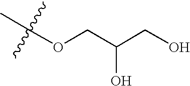

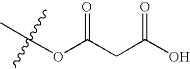

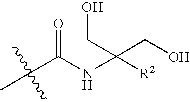

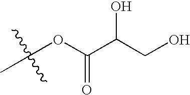

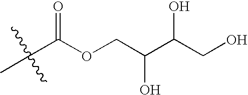

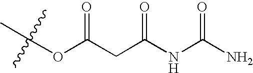

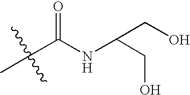

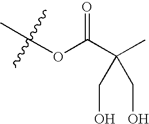

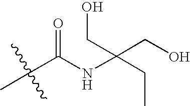

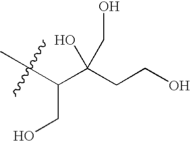

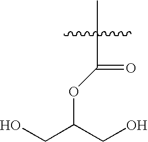

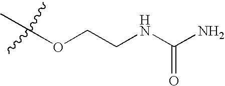

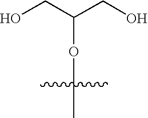

Surfactants of the present invention can be synthesised by known methods from starting materials that are known, are themselves commercially available, or may be prepared by standard techniques of organic chemistry used to prepare corresponding compounds in the literature.

For example, urea based surfactants can be prepared by coupling an amine with a selected tail group and then further reacting the alkylamine to form the urea derivative. Glycerol derivatives can be prepared by reaction of the appropriate organic acid with glycerol as the alcohol; protection/deprotection of the various alcohol groups can be utilised to achieve regio-specific coupling to form the surfactant. Glycerate derivatives can be prepared by treating an active glyceric acid derivative with an alcohol containing the tail group of interest.

The above-described reactions can take place at varying temperatures depending, for example, upon the solvent used, the solubility of any reactants and intermediates. Preferably, however, when the above reaction is used, it takes place at a temperature from about 0° C. to about 100° C., preferably at about room temperature. The time required for the above reactions also can vary widely, depending on much the same factors. Typically, however, the reaction takes place within a time of about 5 minutes to about 24 hours.

The product is isolated from the reaction mixture by conventional techniques, such as by precipitating out, extraction with an immiscible solvent under appropriate pH conditions, evaporation, filtration, crystallisation, or by column chromatography on silica gel and the like. Typically, however, the product is removed by either crystallisation or column chromatography on silica gel, followed by purification on reverse phase HPLC if required.

Precursor compounds can be prepared by methods known in the art. Other variations and modifications of this invention using the synthetic pathways described above will be obvious to those skilled in the art.

It is thought that the combination of a relatively small polar head group and a tail that provides a wedge-shaped molecular geometry results in the surfactants of the present invention forming cubic or reverse hexagonal phases in excess water. Branched alkyl chains such as those based on (3,7,11-trimethyl)dodecane(hexahydrofarnesol) and (3,7,11,15-tetramethyl)hexadecane(phytanol) are particularly useful tail groups for the purposes of the present invention. Aliphatic chains that include one or more cis-double bonds such as those based on oleyl or linoleyl chains have also been found to be useful tail groups.

Preliminary assessment of the phase behaviour of a selected compound was conducted using the ‘flooding’ technique. The flooding technique involves placing the compound between a coverslip and microscope slide and introducing water to the sample to establish a water concentration gradient through the sample. This technique is well described in the art for the purpose of identifying which lyotropic phases a surfactant will form in the presence of water, and in what order the phases appear with increasing water content, however it does not provide any details about the water content at the boundaries between phases. If the experiment is conducted on a hot stage, the temperature range over which the particular lyotropic phases exist can also be determined. The phase behaviour can be observed under normal or cross-polarised light using an optical microscope. The identity of the phase is revealed to those skilled in the art by the unique textures observed under crossed polarised light, and the sequence of observed phases through the sample. For the purpose of the present invention it was especially useful for identifying which phase was present at the boundary with excess water.

In addition to this preliminary screening method, two methods were used to quantify the phase boundaries in terms of composition. The first method involves preparation of surfactant and water mixtures in known ratios, sealed in ampoules, and determination of the phase or phases formed at equilibrium. The second method involves the simultaneous use of the flooding experiment combined with near-infrared determination of water content at various points along the concentration gradient, which can be correlated with the phase type.

Further structural evaluation of hexagonal or cubic phases of the lyotropic phases can be performed using Small Angle X-ray Scattering (SAXS) studies, visualisation of the dispersed structures by light microscopy and electron microscopy, for example cryo-Transmission Electron Microscopy (cryo-TEM), Nuclear Magnetic Resonance spectroscopy (NMR), light scattering studies for the measurement of particle size distributions, Differential Scanning Calorimetry (DSC) or a combination of any two or more of the above techniques. In most cases, structural evaluation can be conducted on both bulk samples of the lyotropic phase, and on colloidal dispersions of the bulk lyotropic phase.

The present invention is principally concerned with binary and pseudo-binary systems in which the surfactant is mixed with a polar liquid such as water in the case of binary systems, whilst in a pseudo-binary systems, other water- or oil-soluble components may be present. Ternary systems may also be produced with these surfactants by addition of a non-polar solvent to the surfactant-water mixture. It should be appreciated that the present invention may in some cases provide access to a particular lyotropic reverse phase as a binary system, which hitherto has only been accessible through a ternary system with currently known surfactants.

Compositions containing reverse lyotropic phases formed from surfactants of the present invention may be prepared using water as the hydrophilic liquid component. The compositions may also contain additives, such as, but not limited to, stabilisers, preservatives, colouring agents, buffers, cryoprotectants, viscosity modifying agents, other surfactants of the present invention, and other functional additives.

Advantageously, the thermodynamic stability of the reverse phases to dilution in excess aqueous solution means that they can be dispersed to form colloidal particles of the reverse lyotropic phase. Colloidal particles containing cubic phase or hexagonal phase are sometimes referred to as cubosomes or hexosomes, respectively. In each of these phases, the non-polar tails of the * surfactants comprise the internal hydrophobic domains of the reverse lyotropic phase, while the hydrated head groups occupy the interface between the hydrophobic domain and the internal and external aqueous domains.

The compositions of the present invention may be formed using any suitable process. However, most preferably the process includes the steps of melting the surfactant, if required, and homogenising the molten surfactant in aqueous medium. Alternatively, the composition may be formed in any manner by addition of the aqueous component to the molten, liquid or liquefied surfactant, which may or may not contain other solutes.

The reverse lyotropic phases may contain a solute compound that is included within the reverse lyotropic phase. The solute in this case may reside in the hydrophobic domain, the hydrophilic domain, or in the interfacial region of the reverse phase, or the solute may be distributed between the various domains by design or as a result of the natural partitioning processes. If the solute is amphiphilic it may reside in one or any number of these domains simultaneously. Importantly, the ability to load solutes into the various regions may be of particular advantage in the use of the surfactants of the present invention.

Potential solutes may include but are not limited to diagnostic agents, polymerisation monomers, polymerisation initiators, proteins and other polypeptides, oligonucleotides, denatured and non-denatured DNA, radioactive therapeutic agents, sunscreen active constituents, skin penetration enhancers, skin disease therapeutic agents, transdermally active compounds, transmucosally active compounds, skin repair agents, wound healing compounds, skin cleansing agents, degreasing agents, viscosity modifying polymers, hair care actives, agricultural chemicals such as fungicides and pesticides, fertilisers and nutrients, vitamins and minerals, explosives or detonatable materials and components thereof, mining and mineral processing materials, surface coating materials for paper, cardboard and the like, among others.

In order for compositions containing reverse lyotropic phases to be of use commercially, it is preferable that the phases or colloidal particles are stable for an extended period of time at the storage temperature. For the present purposes. ‘stable’ means that the reverse lyotropic phases do not undergo a detrimental phase change due to storage conditions or chemical degradation. Alternatively, they must be amenable to other processes to increase stability, such as solidification or gelation of the surrounding medium, freezing, freeze-drying or spray-drying. Further, the formation of the reverse phase by addition of a precursor solution containing the surfactant and other components, such as a hydrotrope, to the aqueous phase is also considered a method to circumvent stability issues. Another consideration in terms of the stability of the phases is that they must also be stable at a working temperature. The working temperature will of course depend on the application for which the reverse lyotropic phases are used. For ease of storage the reverse lyotropic phases are preferably stable at room temperature.

In terms of stability, the use of surfactants which display high transition temperatures may be of particular benefit, as solidification by reducing the temperature below the temperature of formation of the reverse lyotropic phase can trap the aqueous domains and water soluble solutes in the solid matrix. The solid. matrix may impart additional stability on the system. On heating to the transition temperature, the reverse lyotropic phase may be reformed, thereby allowing function of the reverse phase, or dispersion of reverse lyotropic phase as intended for the application.

Preferably the reverse lyotropic phases of the present invention form within a temperature range of about −100° C. to about 150° C.

In phases formed by surfactants of the present invention the bicontinuous cubic phase has a structure in which a surfactant bilayer separates an inner aqueous volume from an outer one. The bilayer membrane is multiply folded and interconnected. The hexagonal phase consists of rod-like micelles, packed in a hexagonal array, in the surfactant matrix. These structures are well known and described in detail in the surfactant phase behaviour literature.

It is the particular geometry of the surfactants of the present invention that determines the type of arrangement that the molecules adopt at the interface between the hydrophilic and hydrophobic domains, and the subsequent thermodynamically stable phase produced. There is a strong link between the formation of lamellar phase and bicontinuous cubic phase, with the latter usually observed as the intermediate phase between the former and a more hydrophilic water-rich phase as the water content is increased. However, the surfactants of the present invention are not readily soluble in water and hence do not undergo a transition to a more hydrophilic phase with increasing water content. Instead, the excess water is not incorporated at all but exists as a phase separated domain. Likewise for the reverse hexagonal phase, no transition is evident to a more hydrophilic phase due to the finite swelling with water in the hexagonal phase, and the low solubility of the surfactant in water dictates that an excess water phase is produced rather than a phase change to a more hydrophilic homogeneous system. This provides the property of the surfactants of the invention that the reverse lyotropic phases, or the bicontinuous cubic phase will exist in excess water and not undergo a phase change on dilution.

Many of the surfactants of the present invention form a reverse lyotropic phase spontaneously on contact with water at room temperature. Typically as the temperature is increased, the cubic or reverse hexagonal phase begins to slowly melt and mobility is often observed within the phase. On continued heating the sample eventually reaches a temperature at which all liquid crystalline structure is destroyed, leaving an isotropic surfactant-rich phase, and excess water present. On cooling the cubic or reverse hexagonal phase typically reappears, and some supercooling of the phases can be apparent in the temperature of reappearance.

It will be appreciated that a problem with some liquid crystal phases is that the phase changes upon dilution of the solution. For many applications for which they are used it is preferable to have a stable phase that does not change upon dilution with solvent. It has been found that the liquid crystalline phases formed from surfactants of the present invention do not change phase upon solvent dilution.

Preparations of the invention for utility may be of the following two principal forms, although other forms may be required depending on the application.

The first form is the bulk reverse phase, where the entire aqueous component may or may not be incorporated into the reverse lyotropic phase. Preparation of the bulk phase may involve the simple mixing of the surfactant component containing any required solutes, with the aqueous component in a blender, mixer, jet-mixer, homogeniser and the like. The use of a co-solvent that is subsequently removed partly or completely by natural evaporation or under vacuum, or by heating or other means, may allow for easier processing to achieve the bulk reverse phase sample. Alternatively, the solvent may remain as part of the system, if required. Temperature control can also be utilised to facilitate the mixing process, by alteration of the phase behaviour of the mixture, and hence its rheological properties.

The second form is the case in which there is an excess of aqueous solution added to the mixture. As the bulk reverse phase is stable to dilution in excess water, a dispersion of particles of the reverse phase in aqueous solution may be obtained. Aqueous dispersions of the reverse lyotropic phases are obtained by two principal methods, by fragmentation of the homogenous bulk reverse phase, or by in situ formation of the liquid crystal from a dispersion of the surfactant into water, although these are not limiting examples. The fragmentation procedure involves preparation of the bulk reverse phase in the presence of sufficient aqueous phase to form the primary lyotropic phase without excess water present.

Optionally any solute to be carried within the liquid crystalline phase may be added dissolved in either the hydrophobic surfactant component or the hydrophilic aqueous component. The bulk reverse lyotropic phase is then added to a second aqueous solution, which may or may not be identical to the aqueous phase used to form the primary lyotropic phase, and the mixture homogenised by means of a high energy mixer. The resulting coarse dispersion may then be further processed to reduce the size of the dispersed particles by passing the coarse dispersion through a high-pressure homogeniser. Homogenisation conditions are tailored to obtain a mean particle size required for the intended application; with this process it is possible to achieve average particle sizes in the sub-micron size region, often less than 200 nanometres in diameter. The temperature of the process may be important in some instances and can be controlled by utilising thermally jacketed equipment.

Alternatively the particle of reverse lyotropic phase may be prepared in situ, by the addition of the surfactant, possibly dissolved in a suitable hydrotrope, into an aqueous solution under high shear mixing to achieve the coarse dispersion. The choice of hydrotrope may in some cases reduce the energy required to produce a stable coarse dispersion. Subsequent processes to reduce the particle size may be applied as above. The quality and colloidal stability of the dispersions is monitored by particle size analysis and visual observation of instability initially and over time after storage under conditions of interest.

The dispersion of surfactants of this invention which exhibit high melting points is conducted in the same manner as described above, with extra attention being paid to temperature control. Their use in areas where protection of the internal aqueous domains of the particle is required at moderate temperatures, but release of their contents at high temperatures is of particular importance for dispersions of these surfactants.

Compositions of the present invention may be subjected to further treatment processes to render them suitable for use in a particular application. For example, compositions may be sterilised by means of an autoclave, sterile filtration, or radiation techniques.

Colloidal particles or compositions containing them may be further stabilised using a stabilising agent. A variety of agents suitable for this purpose are commonly used in other colloidal systems and may be suitable for the present purposes. For example, poloxamers, alginates, amylopectin and dextran may be used to enhance stability. Addition of a stabilising agent preferably does not affect the final structure or the physical properties of the particles or compositions. More importantly the addition of the stabiliser preferably does not alter the reverse lyotropic phase in contact with excess aqueous phase.

Compositions of the present invention may also be modified by the addition of additives, such as, but not limited to glycerol, sucrose, phosphate buffers and saline in relevant concentrations, to the aqueous medium without changing the principle structure of the particles.

Dispersions of reverse lyotropic phase, including bicontinuous phases are expected to find utility when the bulk material needs to be pumped or handled in some manner in industrial processes, or where a very high surface area is desirable, such as in interfacial polymerisation processes, or as a reaction quencher.

The water resistant properties of the phases formed by the surfactants of the present invention provide for the use of the materials as water resistant coatings and lubricants, where resistance to weathering and/or aqueous environments is required for function or to prolong the life-time of the materials. Application as a coating for paper and cardboard may provide benefits over the currently employed fat- and wax-based coatings, or the reverse phase could function as a carrier for more permanent coating components. The potential to spray the dispersions of the current invention would provide processing benefits for these types of applications.

The formulation of explosives for the mining industry is another potential application of these materials, as the formulation of explosives requires the intimate contact of an organic solution (as the fuel) and an aqueous solution (containing a water-soluble oxidising agent). The contact in the current inventions is significantly more intimate than in the currently utilised emulsion formulations. The special application of the present invention to the field of explosives can be recognised from an understanding that the application of explosives in the mining industry if often under extremely damp, wet conditions.

The immobilisation of enzymes and proteins within the reverse lyotropic phase structure is useful, as the interior environment of the reverse lyotropic phase may be controlled to minimise denaturing or degrading of the solute.

The reverse phases and dispersions thereof may also be used as biosensors a change in lyotropic phase on binding of a target molecule or antigen may be used as the transduction mechanism for detection.

Application of the present invention in the fields of polymerisation, reaction control and controlled crystallisation are particularly of interest due to the small particle size and high surface area of the dispersion of these materials. The ability to load reagents with quite differing physico-chemical properties into the different compartments of the invention is of special importance to these applications. As such the invention would be particularly suited to dispersions of two or more reactants into the various compartments of the invention, and introduction of a catalyst or initiator to the external aqueous solution. Alternatively, the catalyst may be included in one of the compartments and a reactant introduced later via the external aqueous solution. In any case, the potential as a site of controlled reaction or polymerisation is an important potential utility of the bulk reverse lyotropic phase and dispersions thereof prepared from these amphiphiles. Controlled crystallisation of materials within the compartments of the phases formed by this invention, allows for templating or restricting the size and shape of novel particles thereby produced.

The area of cosmetics, hair and skin care are also targets for the utility of the materials of the present invention. Again, the ability to load agents with differing properties is important in these utilities. The ability to prepare creams, gels, foams, mousses, oils, ointments and the like using these materials, has potential benefits over traditional materials due to their water resistance, and possible low dermatological irritability. As such, products for haircare applications, topical treatment of antibacterial or antifungal infections, psoriasis and the like, are uses of the current invention.

Because the materials are expected to produce breakdown products with very low oral toxicity, then the application of the materials in food products such as emulsions, dispersions, jellies, jams, dairy products like ice cream and yoghurt, is also expected to be possible. The special rheological properties of these amphiphiles when added to water may be of particular interest for their use as rheology and phase modifiers for these types of systems. Similarly, the materials may be utilised in the formulation of vitamin and mineral supplements, and the like.

### DESCRIPTION OF PREFERRED EMBODIMENTS OF THE INVENTION

Preferred embodiments of the invention will now be described by way of the following non-limiting examples.

### EXAMPLE 1

### 1-(3,7,11,15tetramethyl-hexadecyl)-1-(2-hydroxyethyl) urea

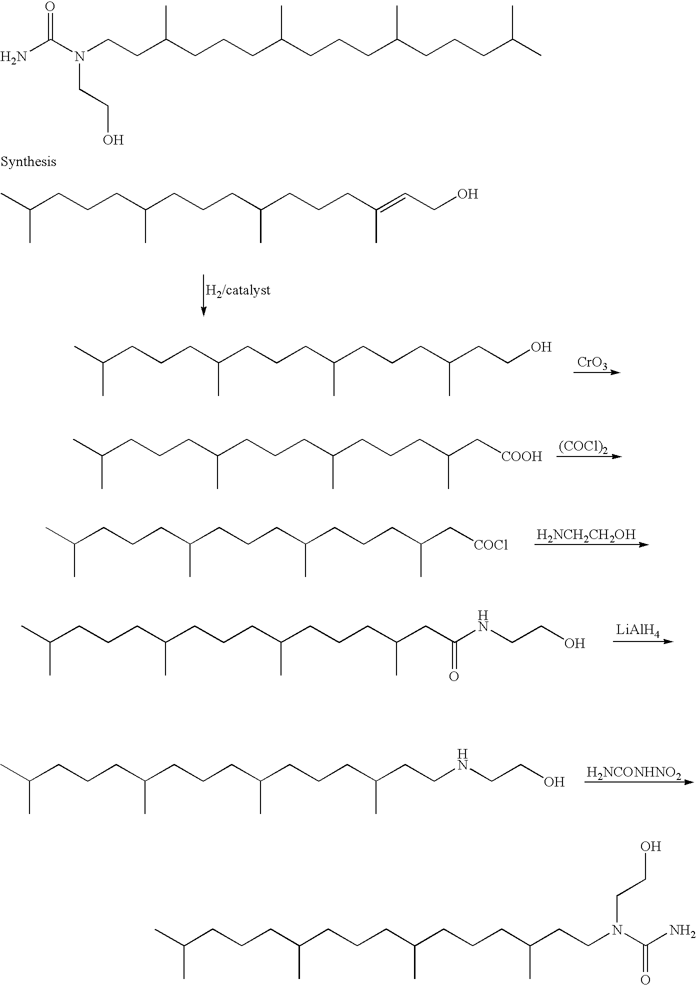 Chemical Characterisation—Elemental Analysis

Calc: C 71.82, H 12.58, N 7.28, O 8.32 Anal: C 71.48, H 12.44, N 6.81, O 9.27

Chemical Characterisation—NMR

¹H NMR m, δ0.78-0.93, 15H hexadecyl CH₃; m, δ0.96-1.65, 24H hexadecyl CH₂+hexadecyl CH; m, δ3.15-3.27, 2H, CO₂—CH₂; t, δ3.39, J 4.85 Hz, NHCH₂CH₂OH; t, δ3.76, J 4.85 Hz, NHCH₂CH₂OH; v br s, δ4.66 2H, N—H; v br s, δ5.35 1H, N—H.

Physical Properties

The compound is a pale yellow oil at room temperature.

Lyotropic Behaviour

At 20° C. water fingers inwards and a reverse hexagonal phase develops instantaneously at the interface, broadening slowly on standing for 20 minutes. On heating, at 50.9° C. the hexagonal phase begins to melt, converting to a mobile isotropic phase, and the sample is completely isotropic by 58.1° C. The mobile isotropic phase remains up to 100° C. On rapid cooling the hexagonal phase redevelops at 51.1° C.

### EXAMPLE 2

### 1-(3,7,11,15-tetramethyl-hexadecyl)-3-(2-hydroxyethyl) urea

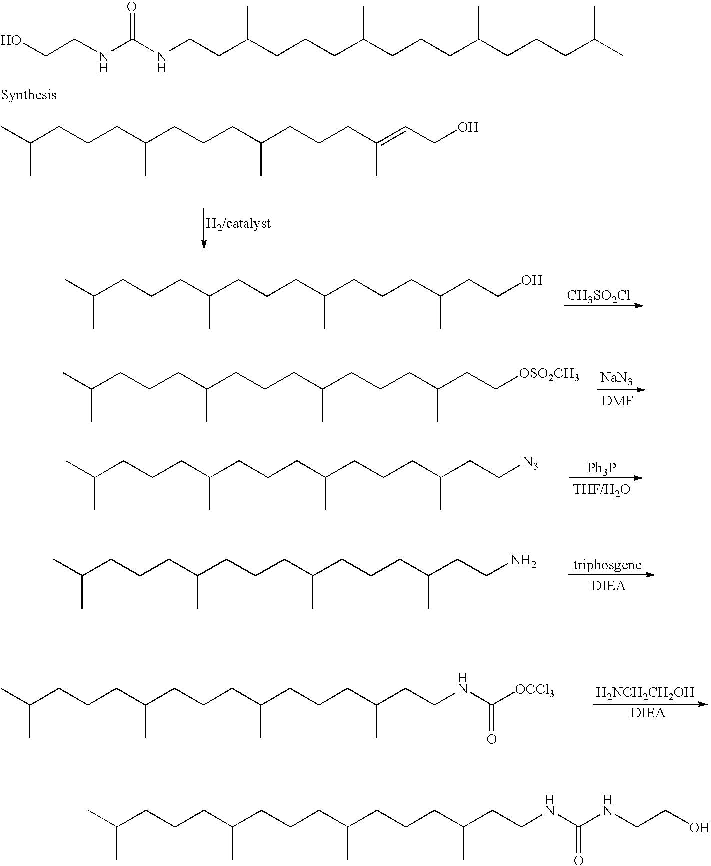 Chemical Characterisation—Elemental Analysis

Calc: C 71.82, H 12.58, N 7.28, O 8.32 Anal: C 71.84, H 12.77, N 7.38, O 8.01

Chemical Characterisation—NMR

¹H NMR m, δ0.76-0.94, 15H hexadecyl CH₃; m, δ0.94-1.60, 24H hexadecyl CH₂+hexadecyl CH; m, δ3.03-3.23, 2H, CO₂—CH₂; t, δ3.30, J 4.7 Hz, NHCH₂CH₂OH; t, δ3.66, J 4.7 Hz, NHCH₂CH₂OH; v br s, δ4.68 3H.

Physical Properties

Colourless oil at room temperature.

Lyotropic Behaviour

This surfactant forms a reverse hexagonal phase at the interface with water for a broad temperature regime, commencing from at below 8° C. and melting completely at 58° C. Commencing at 40.4° C., the reverse phase melts slowly, forming an isotropic phase adjacent to the interface and this is highly mobile and expands outwards. The sample is completely isotropic by 57.3° C. The reverse hexagonal phase recrystallises at 44.1° C. on cooling.

### EXAMPLE 3

### 3,7,11,15Tetramethyl-hexadecyl urea

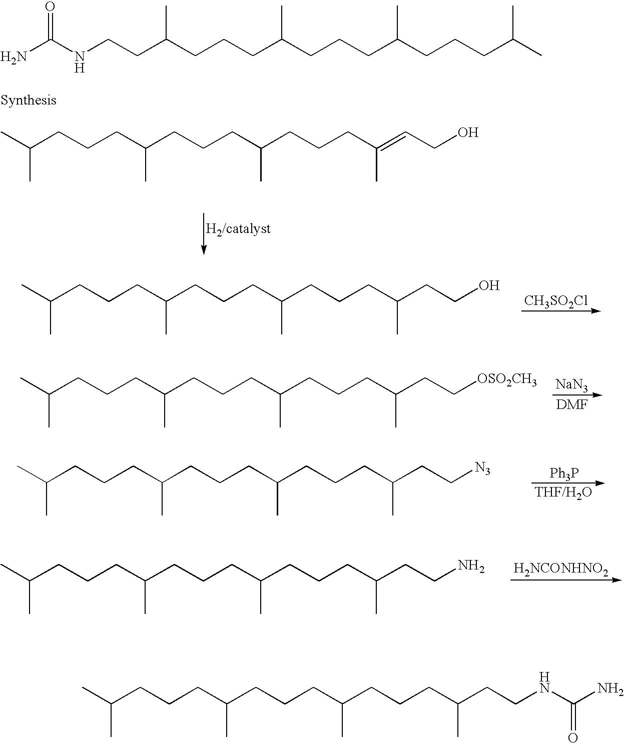 Chemical Characterisation—Elemental Analysis

Calc: C 74.06, H 13.02, N 8.22, O 4.70 Anal: C 73.79, H 12.83, N 8.11, O 5.97

Chemical Characterisation—NMR

¹H NMR m, δ0.78-0.93, 12H hexadecyl CH₃; m, δ0.93-1.60, 24H hexadecyl CH₂+hexadecyl CH; m, δ3.00-3.23, 2H, CO₂—CH₂; v br s, δ4.66, 2H; v br s, δ5.35, 1H.

Physical Properties

The compound forms a thermotropic liquid crystal on standing at room temperature, which melts at 60.6-65.6° C.

Lyotropic Behaviour

At 25° C. a reverse hexagonal phase forms along the interface of the surfactant with the water, with an isotropic band between it and the unchanged surfactant. The position of the interface of the phase with water does not move on when held at 25° C. Fluidity was observed in the isotropic band and small spherical bubbles in both mesophases was noted. At 49.6+ C. the isotropic band begins to replace the crystal and develops rapidly as the temperature is raised. The surfactant core is isotropic by 54.90C. At 72.6° C., a melting of the reverse hexagonal phase to an isotropic liquid at the interface with water commences, and is complete by 82.1° C.

### EXAMPLE 4

### 3,7,11-Trimethyl-dodecyl urea

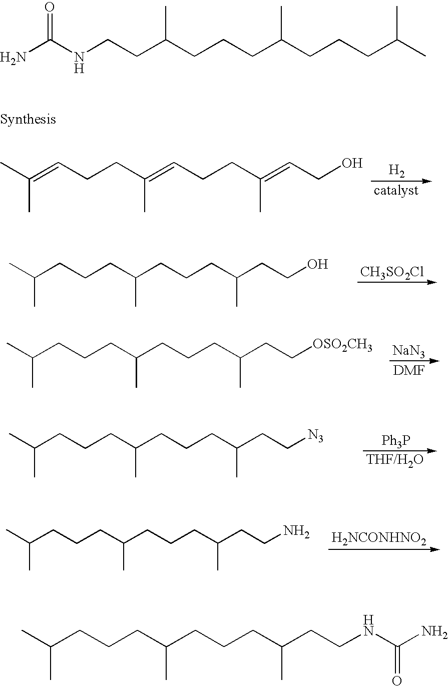 Chemical Characterisation—Elemental Analysis

Calc: C 71.06, H 12.67, N 10.36, O 5.92 Anal: C 71.41, H 12.38, N 10.37, O 5.84

Chemical Characterisation—NMR

¹H NMR m, δ0.77-0.92, 12H dodecyl CH₃; m, δ0.92-1.65, 17H dodecyl CH₂+dodecyl CH; m, δ3.15-3.27, 2H, CO₂—CH₂; v br s, δ4.66 2H, N—H; v br s, δ5.35 1H, N—H

Physical Properties

Clear viscous mesomeric liquid at room temperature. Liquid crystalline melting point 61-62.5° C.

Lyotropic Behaviour

On contact of water with the viscous oily surfactant at 30° C., there is rapid ingress of water into the oil and a reverse hexagonal phase texture appears immediately at the interface in the oil halting further water ingress. The reverse hexagonal phase is clearly apparent between 30° C. and 50° C. Some dynamic effects at the interface with water occur at 55° C., characterised by apparent melting and re-growth of the hexagonal phase. Significant melting and re-growth occurs at 60° C., with complete melting of the reverse hexagonal phase occurring at >70° C.

### EXAMPLE 5

### 2,3-Dihydroxypropionic acid octadec-9-enyl ester

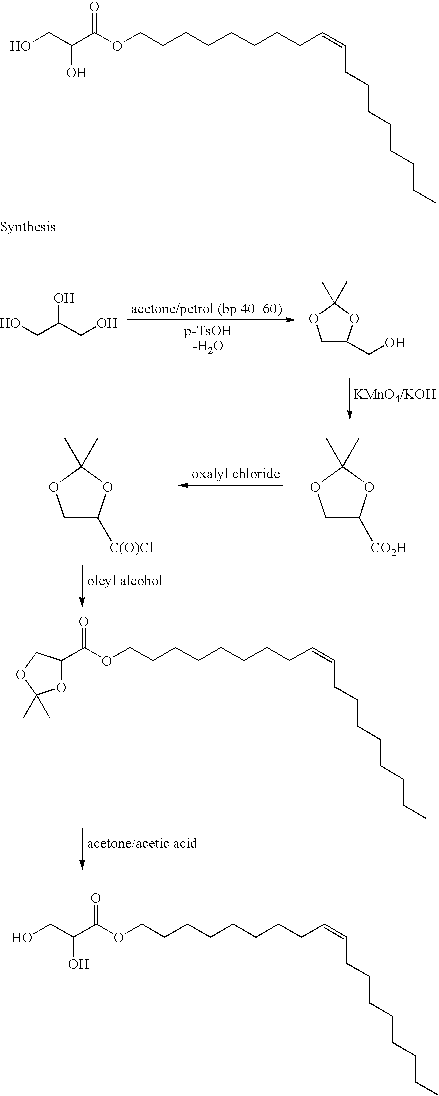 Chemical Characterisation—Elemental Analysis

Calc: C 71.35, H 10.55, O 18.10 Anal: C 70.39, H 10.92, O 18.69

Chemical Characterisation—NMR

¹H NMR δ(CDCl₃) sl br t, δ0.88, 3H, splitting 6.3 Hz, oleyl CH₃; m, δ1.2-1.45, 22H oleyl CH₂; m, δ1.55-1.75, 2H, CH₂CH₂CO₂; m, δ1.92-2.1, 4H, CH₂CH═CHCH₂; v br s*, δ2.05-2.45, 1H, OH; v br s*, δ3.05-3.40, 1H, OH; dd, δ3.83, 1H, J −11.7 Hz 3.7 Hz, glyceryl C3-H; dd, δ3.90, 1H, J −11.7 Hz 3.3 Hz, glyceryl C3-H; t, δ4.22, 2H, J 6.7 Hz, oleyl CH₂O; dd, δ4.26, 1H, J 3.7 Hz 3.3 Hz, glyceryl C2-H; m, 2H, δ5.3-5.4, CH═CH. The resonances at 2.2 and 3.2 disappear on D₂O treatment

Physical Properties

Partially crystalline wax at 23° C. Viscosity drops at 30° C. The crystals melt at 30 to 35° C.

Lyotropic Behaviour

On addition of water at 30° C., a large ingress of water occurs into the surfactant, and initially forms a reverse hexagonal phase at the interface with water, but on holding at 30° C. an isotropic viscous cubic phase appears at the interface with water. The cubic phase boundary with the hexagonal phase moves to the pure surfactant region as the temperature is raised from 30-55° C. At 55-60° C., the isotropic cubic phase narrows slightly, and at 65° C. the hexagonal texture starts to melt. At 70° C., the isotropic phase has disappeared, and further melting of the hexagonal phase is evident; this process continues until a single isotropic non-viscous liquid is formed at 80° C. This process is reversible—lowering the temperature to 77° C. causes the hexagonal texture to reappear, and lowering further to 40° C. results in the isotropic phase reforming.

### EXAMPLE 6

### 2,3-Dihydroxypropionic acid 3,7,11,15-tetramethyl-hexadecyl ester

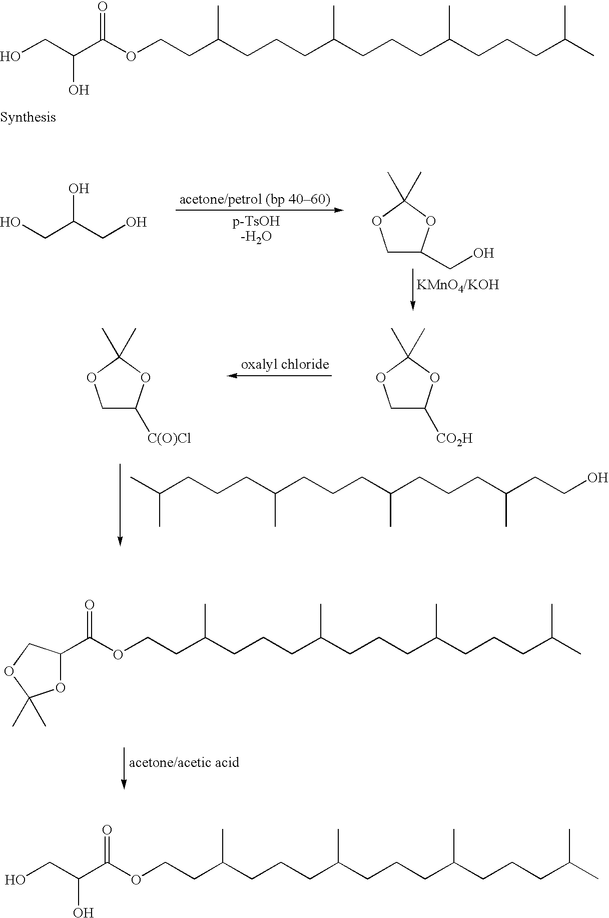 Chemical Characterisation—Elemental Analysis

Calc: C 71.45, H 11.99, O 16.55 Anal: C 70.78, H 12.24, O 16.98

Chemical Characterisation—NMR

¹H NMR m, δ0.78-0.93, 15H hexadecyl CH₃; m, δ0.93-1.80, 24H hexadecyl CH₂+hexadecyl CH; dd, δ2.13, 1H, J 8.5 Hz 4.6 Hz, glyceryl C3-OH; d, δ3.16, 1H, J 4.6 Hz, glyceryl C2-OH; ddd, δ3.83, 1H, J −11.4 Hz 4.1 Hz 8.5 Hz, glyceryl C3-H; ddd, 1 H, δ3.90, J-11.4 Hz 3.4 Hz 4.8 Hz, glyceryl C3-H; ddd, δ4.27, 1 H, J 4.6 Hz 4.1 Hz 3.4 Hz, glyceryl C2-H; t, δ4.22, 2H, J 6.7 Hz, CO₂—CH₂.

After treatment with D₂O m, δ0.78-0.93, 15H hexadecyl CH₃; m, δ0.93-1.80, 24H hexadecyl CH₂+hexadecyl CH; dd, δ3.83, 1H, J −11.4 Hz 4.1, glyceryl C3-H; dd, 1H, δ3.90, J-11.4 Hz 3.4 Hz; glyceryl C3H; dd, δ4.27, 1H, J 4.1 Hz 3.4 Hz, glyceryl C2-H; t, δ4.22, 2H, J 6.7 Hz, CO₂—CH₂. The resonances previously at 2.13 and 3.16 have disappeared.

Physical Properties

Pale yellow oil at room temperature.

Lyotropic Behaviour

A reverse hexagonal phase forms spontaneously at the boundary between the surfactant and excess water at room temperature. On heating, a slow onset of melting of the reverse hexagonal phase begins at ˜40° C., and water observed to finger its way into the reverse hexagonal phase structure. The entire sample appears isotropic when 48° C. is reached.

### EXAMPLE 7

### 3,7,11,15-tetramethyl-hexadecanoic acid (1,1-bis-hydroxymethyl-ethyl)-amide

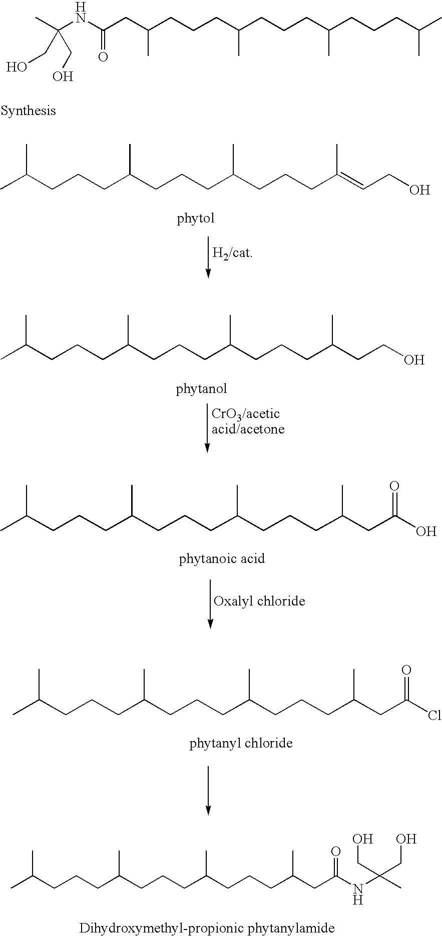 Chemical Characterisation—NMR

¹H NMR sl br d, δ0.84, 6H, splitting 6.3 Hz, CH₃; d, δ0.86, 6H, splitting 6.6 Hz, CH₃; d, δ0.94, 3H, splitting 6.2 Hz, CH₃; m, δ0.97-1.42, 21H, chain CH₂+CH; s, 1.23, 3H, CH₃CH—N; m, δ1.40-1.63, 1H, C(3)-H; m, δ1.85-20.7, 1.45H, CH₂—N; m, δ2.15-2.34, 0.55H, CH₂—N; br s, δ3.47, 2H, OH; d, δ3.60, 2H, J 11.5 Hz, CCH₂OH; d. δ3.74, 2H, J 11.5 Hz, CCH₂OH; br s, δ6.02, 1H, NH.

Physical Properties

Pale yellow viscous oil with flecks of crystalline material at room temperature.

Lyotropic Behaviour

At 10-15° C. this surfactant rapidly develops an isotropic phase at the interface with water, and a hexagonal phase between it and the unchanged surfactant. There was no change in the position of the interface with water as the sample was kept at 23° C. for 30 mins, and the 2 regions develop very slowly inwards, indicating that they are reverse lyotropic phases. In some locations water fingered into the oil and dendritic features are observed along the water perimeter. The isotropic band appears viscous and no fluidity was observed within the phase. Entrapped bubbles are non-spherical.

The hexagonal phase began to melt at 25.5° C. and is completely isotropic by 26.7° C. The hexagonal phase, on melting, appears to form a second isotropic phase. The boundary is indicated by a refractive index change. At 32.9° C. beading occurs in the isotropic phase in contact with water. As the sample is maintained at 32.9° C. for 20 mins, the formerly-hexagonal isotropic area expands outwards towards the water interface consuming the viscous isotropic region. At 34.4° C. the two isotropic phases appear to convert to a single isotropic phase which is much more mobile. As the temperature increases up to 95° C., globules of the isotropic phase separate into the adjacent water phase.

### EXAMPLE 8

### 1-(2-Hydroxyethyl)-3-(cis-octadec-9-enyl) urea

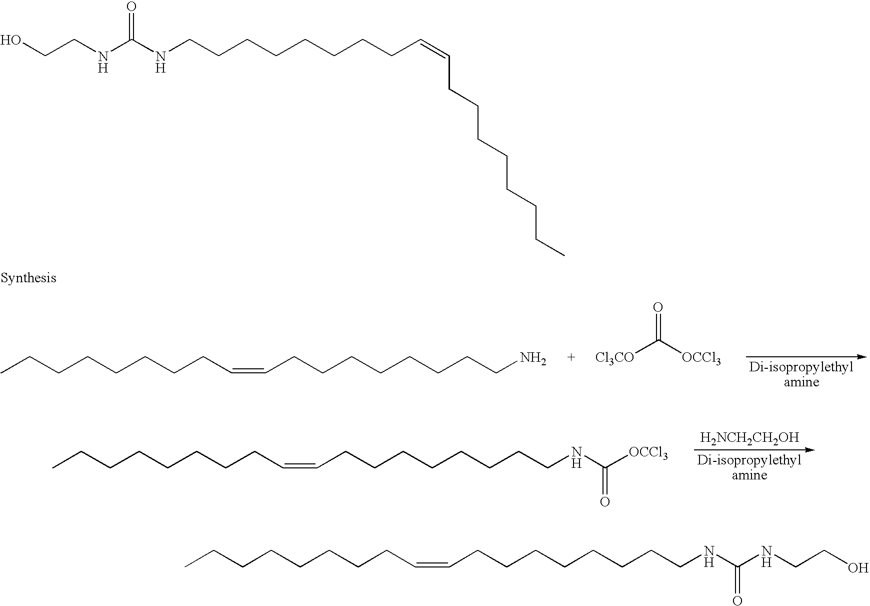 Chemical Characterisation—NMR

¹H NMR sl br t, δ0.88, 3H, splitting 6.4 Hz, oleyl CH₃; m, δ1.17-1.43, 22H, oleyl CH₂; m, δ1.43-1.63, 2H, oleyl CH₂CH₂N; m, δ1.91-2.08, 4H, CH₂CH═CHCH₂; t, δ3.19, 2H, J 7.6 Hz, oleyl CH₂N; t, δ3.36, 2H, J 4.8 Hz, ethyl CH₂N; t, δ3.72, 2H, J 4.8 Hz, ethyl CH₂OH; m, δ5.25-5.43, 1.75H, CH═CH.

Physical Properties

A white crystalline solid with a melting point of 80-84.7° C.

Lyotropic Behaviour

No interaction between the solid surfactant and water occurs on heating until a temperature of 59.5° C. is attained, when there is a gradual development of an isotropic phase in contact with the water. The isotropic band broadens slowly into the surfactant core as the sample is maintained at 62° C. for 10 minutes. At the very edge of the interface, a gel-like consistency is observed, indicating a high viscosity lyotropic phase. There is a slight refractive index difference between the inner (region 2) and outer (region 1) regions of the isotropic band. The outer region expands steadily inwards. No fluidity is apparent within either of these isotropic regions; high viscosity of these regions is suggested by the entrapment of non-spherical bubbles.

At 64.4° C. a lamellar+isotropic (region 3), and another isotropic phase (region 4) developed adjacent to residual surfactant, and expanded inwards. This was indicated by a refractive index difference. Mobility was observed in the inner isotropic phase, indicating a non-viscous phase. By ˜67° C. the sample is completely isotropic with the lamellar phase converted to an isotropic phase which gradually overtook the surfactant core. At 73° C., the initially region 2 slowly expanded and by 83° C. overtook region 3. The refractive index difference between region 1 and 2 are maintained up to high temperature (>98° C.).

### EXAMPLE 9

### cis-octadec-9-enyl biuret

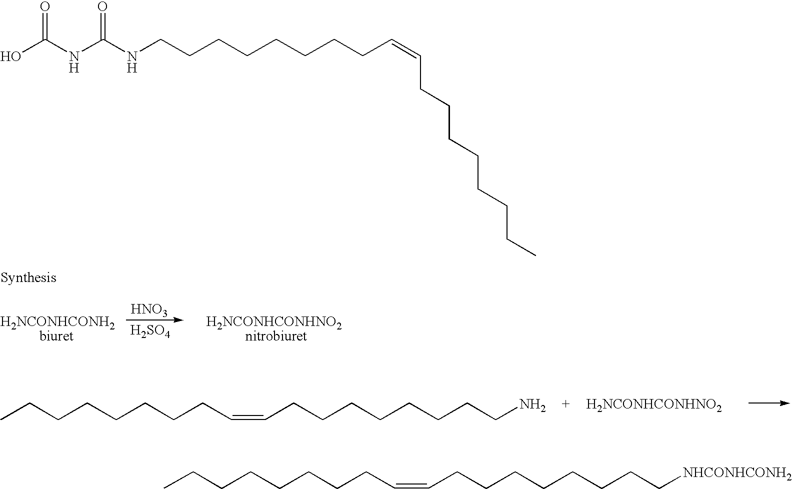 Chemical Characterisation—NMR

¹H NMR sl br t, δ0.88, 3H, splitting 6.5 Hz, oleyl CH₃; m, δ1.17-1.43, 22H, oleyl CH₂; m, δ1.43-1.63, 2H, CH₂CH₂N—; m, δ1.89-2.08, 4H, CH₂CH═CHCH₂; sl br dt, δ3.22, 2H, J 5.6 Hz 6.9 Hz z, oleyl CH₂N; m, δ5.23-5.44, 2, CH═CH.”

Physical Properties

White waxy solid with melting point 100-106° C.

Lyotropic Behaviour

The solid crystalline surfactant was unchanged on heating with water until 85° C. was reached when a hexagonal phase began to form at the interface with water. When the temperature was raised to 87° C., a fluid isotropic phase began to form between the hexagonal phase and the crystals. The hexagonal phase melted at 107° C.

### EXAMPLE 10

### cis-octadec-9enyl urea

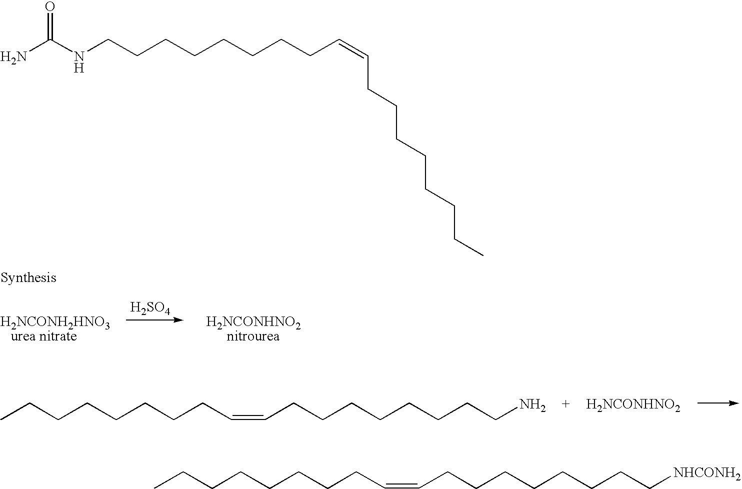 Chemical Characterisation—NMR

¹H NMR sl br t, δ0.88 3H, splitting 6.5 Hz, CH₃; m, δ1.10-1.70, 24H, oleyl-CH₂; m δ1.89-2.12, 4H, CH₂CH═CHCH₂; t δ3.14, 2H, splitting 7.0 Hz, CH₂—NHCONH₂; v br s, δ3.3-4.3, 3H, NHCONH₂; m, δ5.23-5.44, 2H, CH═CH.

Physical Properties

White waxy solid with melting point 68-83° C.

Lyotropic Behaviour

On contact with water there was no change until 61° C. when a reverse hexagonal phase began to form. At 65° C. a fluid isotropic phase began to form between the hexagonal phase and solid urea. As the temperature was further raised, the solid urea first converted to the fluid isotropic phase, and then to the hexagonal phase. All material eventually converted to the hexagonal phase, which melted at 110° C.

### EXAMPLE 11

### cis, cis-octadec-9,12-dienyl urea

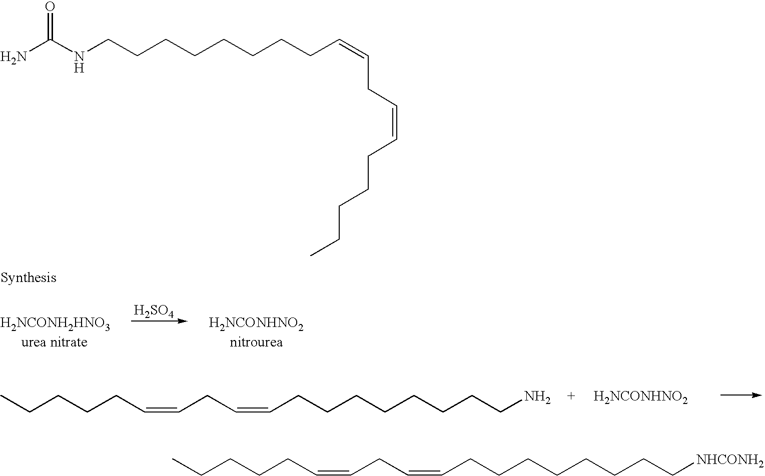 Chemical Characterisation—NMR

¹H NMR sl br t, δ0.89, 3H splitting 6.5 Hz, CH₃; m, δ1.15-1.63, 20H, CH₂; m, δ1.93-2.17, 4H CH₂—CH₂—C═C; sl br t, δ2.78, 2H, splitting 5.5 Hz, C═C—CH₂-C═C; sl br t, δ3.35, 2H, splitting 4.7 Hz, oleyl-CH₂—NH; v br s, δ3.3-4.4, 2.5H, —NHCONH₂; v br s, δ4.5-5.1, 0.9H, NHCONH₂; m, δ5.22-5.42, 4H, CH═CH.

Physical Properties

White waxy solid with melting point 70-79° C.

Lyotropic Behaviour

On contact with water there was no change until 53° C. when a reverse hexagonal phase began to form. At 59° C. a fluid isotropic phase began to form between the hexagonal phase and solid urea. As the temperature was further raised, the solid urea first converted to the fluid isotropic phase, and then to the hexagonal phase. Invasion of water fingers accelerated this process. At 80° C. the solid urea melted, and the rapid invasion of water fingers allowed all material to convert to the hexagonal phase. The hexagonal phase melted at 92-93° C.

### EXAMPLE 12

### Formation of Viscous Lyotropic Phase by Surfactants in the Presence of Water

For the surfactant to be useful it preferably forms a viscous lyotropic phase in the presence of excess water. The lyotropic phase formed by the surfactant in excess water was determined by flooding experiments, in which a small amount of lipid (typically 5 mg) is placed between a glass microscope slide and coverslip and water introduced to the sample by capillary action, with the sample maintained at 40° C. by means of a hot stage. Observation under crossed polarised light at 200× magnification allows identification of the phase formed by the visible birefringent texture, or lack thereof. Table 1 lists the surfactants tested and the lyotropic phase formed on exposure to excess water.

The mass of water incorporated in the lyotropic phase was determined by preparing a 300 mg sample of surfactant in excess water, equilibrating at 40° C., and testing the water content of the lyotropic phase by Karl Fisher titration. These values for the surfactant water combinations tested are also listed in Table 1. Values reported are the mean of three separate samples i standard deviation, unless otherwise indicated.

TABLE 1

| | Phase | |
|---|---|---|
| | formed | % water (w/w) in |
| | in excess | saturated lyotropic |
| Surfactant | water^{a} | phase |
| 2,3-Dihydroxypropionic acid octadec-9- | Hₗₗ | 16.8 ± 3.9 |
| enyl ester | | |
| 2,3-Dihydroxypropionic acid 3,7,11,15- | Hₗₗ | 28.7 ± 2.5 |
| tetramethyl-hexadecyl ester | | |
| 3,7,11-Trimethyl-dodecyl urea | Hₗₗ | 8.1 ± 2.5 |
| 3,7,11,15-Tetramethyl-hexadecyl urea | Hₗₗ | 28.4 ± 2.3 |
| 1-(3,7,11,15-tetramethyl-hexadecyl)-3- | Hₗₗ | 14.3 ± 4.6 |
| (2-hydroxyethyl) urea | | |
| 1-(3,7,11,15-tetramethyl-hexadecyl)-1- | Hₗₗ | ND |
| (2-hydroxyethyl) urea | | |
| 3,7,11,15-tetramethyl-hexadecanoic | Hₗₗ | 23.1 ± 3.2 |
| acid 1-glycerol ester | | |
| 2,3-Dihydroxypropionic acid 3,7,11- | Hₗₗ | 24.7 ± 0.7 |
| trimethyl-dodecyl ester | | |

^{a}Hₗₗ denotes reverse hexagonal phase;
ND = not determined

Finally, there may be other variations and modifications made to the preparations and methods described herein that are also within the scope of the present invention.

1. A compound containing a head group selected from the group consisting of any one of structures (I) to (III): 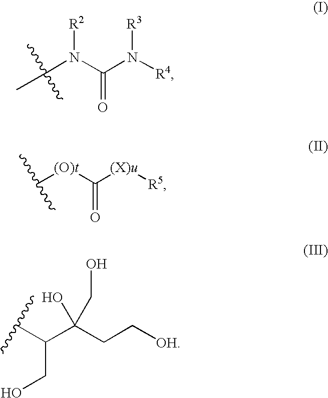
wherein
in structure (I) R² is —H, —CH₂CH₂OH or another tail group,
R³ and R⁴ are independently selected from one or more of —H, —C(O)NH₂, —CH₂CH₂OH, —CH₂CH(OH)CH₂OH,
in structure (II) X is O, S or N,
t and u are independently 0 or 1,
R⁵ is —C(CH₂OH)₂alkyl, —CH(OH)CH₂OH (provided the tail group is not oleyl),
—C(OH)₂CH₂OH, —CH(CH₂OH)₂, —CH₂(CHOH)₂CH₂OH,
—CH₂C(O)NHC(O)NH₂;
and a tail selected from: 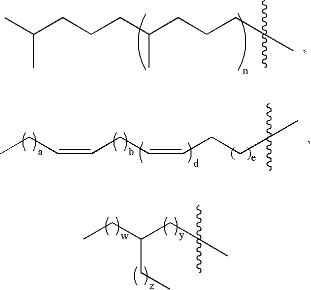
wherein n is an integer from 2 to 6, a is an integer from 1 to 12, b is an integer from 0 to 10, d is an integer from 0 to 3, e is an integer from 1 to 12, w is an integer from 2 to 10, y is an integer from 1 to 10 and z is an integer from 2 to 10.

2. A compound as in claim 1 wherein the tail is selected from the group consisting of (3,7,11-trimethyl)dodecane, (3,7,11,15-tetramethyl)hexadecane, octadec-9-enyl and octadec-9,12-dienyl chains.

3. A compound as in claim 2 wherein the head group is:

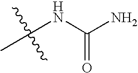

4. A compound as in claim 2 wherein the head group is:

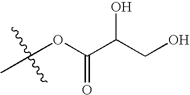

5. A compound as in claim 2 wherein the head group is:

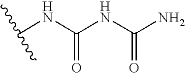

6. A compound as in claim 2 wherein the head group is:

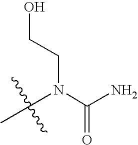

7. A compound as in claim 2 wherein the head group is:

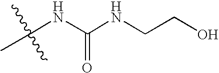

8. A compound as in claim 2 wherein the head group is:

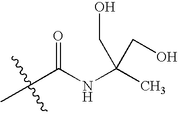

9. A surfactant that forms a lyotropic phase that is stable in excess polar solution, the surfactant containing a head group selected from the group consisting of any one of structures (I) to (V): 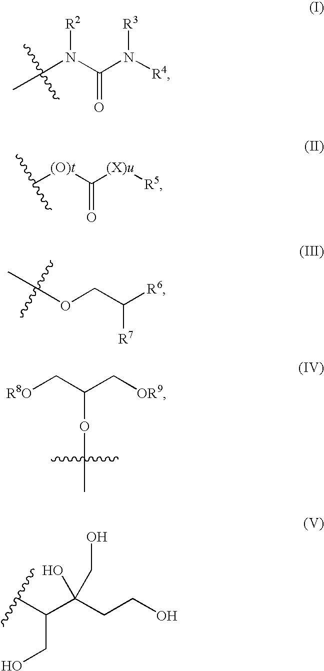
and a tail selected from the group consisting of a branched alkyl chain, a branched alkyloxy chain or an alkenyl chain, and wherein
in structure (I) R² is —H, —CH₂CH₂OH or another tail group,
R³ and R⁴ are independently selected from one or more of —H, —C(O)NH₂, —CH₂CH₂OH, —CH₂CH(OH)CH₂OH,
in structure (II) X is O, S or N,
t and u are independently 0 or 1,
R⁵ is —C(CH₂OH)₂alkyl, —CH(OH)CH₂OH (provided the tail group is not oleyl), —CH₂COOH,
—C(OH)₂CH₂OH, —CH(CH₂OH)₂, —CH₂(CHOH)₂CH₂OH,
—CH₂C(O)NHC(O)NH₂,
in structure (III) R⁶ is —H or —OH,
R⁷ is —CH₂OH or —CH₂NHC(O)NH₂,
in structure (IV) R⁸ is —H or -alkyl,
R⁹ is —H or -alkyl.

10. A surfactant as in claim 9 wherein the tail is selected from: 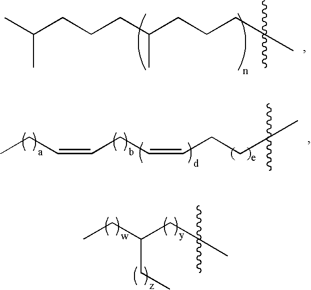
wherein n is an integer from 2 to 6, a is an integer from 1 to 12, b is an integer from 0 to 10, d is an integer from 0 to 3, e is an integer from 1 to 12, w is an integer from 2 to 10, y is an integer from 1 to 10 and z is an integer from 2 to 10.

11. A surfactant as in claim 10 wherein the tail is selected from the group consisting, of (3,7,11-trimethyl)dodecane, (3,7,11,15-tetramethyl)hexadecane, octadec-9-enyl and octadec-9,12-dienyl chains.

12. A surfactant as in claim 11 wherein the head group is:

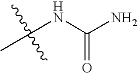

13. A surfactant as in claim 11 wherein the head group is:

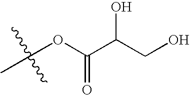

14. A surfactant as in claim 11 wherein the head group is:

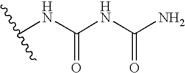

15. A surfactant as in claim 11 wherein the head group is:

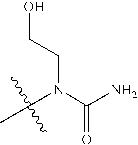

16. A surfactant as in claim 11 wherein the head group is:

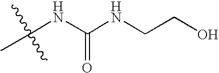

17. A surfactant as in claim 11 wherein the head group is:

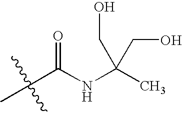

18. A surfactant as in claim 11 wherein me lyotropic phase forms in excess water at a temperature of less than about 150° C.

19. A surfactant as in claim 18 wherein the lyotropic phase that is formed is a bicontinuous cubic liquid crystalline phase.

20. A surfactant as in claim 18 wherein the lyotropic phase that is formed is a reversed hexagonal liquid crystalline phase.

21. A surfactant as in claim 18 wherein the lyotropic phase that is formed does not undergo a transition to a more hydrophilic phase upon addition of excess water.

22. A surfactant as in claim 18 wherein excess water that is added to the lyotropic phase forms a phase separated domain.

23. A surfactant as in claim 18 wherein the lyotropic phase contains a solute that is included within the lyotropic phase.

24. A surfactant as in claim 23 wherein the solute is selected from one or more of the list consisting of diagnostic agents, polymerisation monomers, polymerisation initiators, proteins and other polypeptides, oligonucleotides, denatured and non-denatured DNA, radioactive therapeutic agents, sunscreen active constituents, skin penetration enhancers, skin disease therapeutic agents, transdermally active compounds, transmucosally active compounds, skin repair agents, wound healing compounds, skin cleansing agents, degreasing agents, viscosity modifying polymers, hair care actives, gastric lipase-labile compounds, agricultural chemicals, fertilisers and nutrients, vitamins and minerals, explosives or detonatable materials and components thereof, mining and mineral processing materials, surface coating materials.

25. A composition containing a lyotropic phase formed from a surfactant of claim 9.

26. A colloidal particle consisting of a lyotropic phase of the micellar or liquid crystalline type, formed from a surfactant of claim 9.
